(19) United States
(12) Patent Application Publication
(10) Pub. No.: US 2021/0151126 A1
(43) Pub. Date: May 20, 2021

(54) METHODS FOR FINGERPRINTING OF BIOLOGICAL SAMPLES

(71) Applicant: Lexent Bio, Inc., San Francisco, CA (US)

(72) Inventors: Alexander De Jong Robertson, San Francisco, CA (US); Rohith Kannappan Srivas, San Francisco, CA (US); Timothy Joseph Wilson, San Francisco, CA (US); Neil Peterman, San Francisco, CA (US); Nicole Jacinda Lambert, San Francisco, CA (US); Haluk Tezcan, San Francisco, CA (US)

(73) Assignee: Lexent Bio, Inc., San Francisco, CA (US)

(21) Appl. No.: 17/108,980

(22) Filed: Dec. 1, 2020

Related U.S. Application Data
(63) Continuation of application No. PCT/US2019/035871, filed on Jun. 6, 2019.
(60) Provisional application No. 62/681,642, filed on Jun. 6, 2018.

(51) Int. Cl. G16B 30/00; C12Q 1/6869; C12Q 1/6806

(52) U.S. Cl. CPC G16B 30/00; C12Q 1/6806; C12Q 1/6869

(57) ABSTRACT

The present disclosure provides methods for fingerprinting of biological samples of a subject. In an aspect, the present disclosure provides a method for identifying a sample mismatch, comprising: obtaining a first biological sample comprising a first plurality of nucleic acid molecules from a subject; processing the first plurality to generate a first sample fingerprint comprising a quantitative measure of the first plurality at each of a plurality of genetic loci, wherein the plurality of genetic loci comprises autosomal single nucleotide polymorphisms (SNPs); obtaining a second biological sample comprising a second plurality of nucleic acid molecules from the subject; processing the second plurality to generate a second sample fingerprint comprising a quantitative measure of the second plurality at each of the plurality of genetic loci; determining a difference between the first sample fingerprint and the second sample fingerprint; and identifying the sample mismatch when the difference satisfies a predetermined criterion.

### CROSS-REFERENCE

This application claims the benefit of U.S. Provisional Patent Application No. 62/681,642, filed Jun. 6, 2018, entitled METHODS FOR FINGERPRINTING OF BIOLOGICAL SAMPLES, which is entirely incorporated herein by reference.

### BACKGROUND

The collection and assaying of biological samples obtained from subjects may often encounter challenges with reliable maintenance of sample identity throughout clinical and laboratory processes. For example, biological samples may often be inadvertently swapped in laboratory or clinical settings, thereby resulting in potentially incorrect clinical results if left undetected and uncorrected.

### SUMMARY

Methods for fingerprinting biological samples using panels of genetic loci may require sufficiently deep coverage to obtain genetic information at a desired sensitivity, specificity, or accuracy. For example, deep coverage may be required for a sufficiently high signal-to-noise ratio (SNR) to distinguish between fingerprints generated from different samples. Such samples may be longitudinal samples (e.g., obtained from the same subject at two different time points). Longitudinal samples processed using low-pass sequencing may encounter challenges with (1) correcting matching together samples from different time points and (2) identifying a panel of genetic loci suitable for sample fingerprinting despite relatively low read coverage at any one location.

Methods and systems are provided for generating and comparing fingerprints of biological samples. Sample fingerprints may be generated by sequencing one or more sets of nucleic acid molecules from biological samples obtained from a subject at each of one or more time points. Pairwise comparison of sample fingerprints may be performed to determine whether a sample mismatch (e.g., that the two samples were obtained from different subjects) or a sample match (e.g., that the two samples were obtained from the same subject) is present between the two biological samples from which the sample fingerprints were generated.

In an aspect, the present disclosure provides a method for identifying a sample mismatch, comprising: obtaining a first biological sample comprising a first plurality of nucleic acid molecules from a subject; processing, by a computer, the first plurality of nucleic acid molecules to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); obtaining a second biological sample comprising a second plurality of nucleic acid molecules from the subject; processing, by a computer, the second plurality of nucleic acid molecules to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold. Additionally, in this aspect, the quantitative measure of the first plurality of nucleic acid molecules comprises no more than twelve independent measures of the first plurality of nucleic acid molecules.

In another aspect, the present disclosure provides a method for identifying a sample mismatch, comprising: obtaining a first biological sample comprising a first plurality of nucleic acid molecules from a subject; processing, by a computer, the first plurality of nucleic acid molecules to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); obtaining a second biological sample comprising a second plurality of nucleic acid molecules from the subject; processing, by a computer, the second plurality of nucleic acid molecules to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold. Additionally, in this aspect, the autosomal single nucleotide polymorphisms comprise simple single nucleotide polymorphisms.

In another aspect, the present disclosure provides a method for identifying a sample mismatch, comprising: obtaining a first biological sample comprising a first plurality of nucleic acid molecules from a subject; processing, by a computer, the first plurality of nucleic acid molecules to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); obtaining a second biological sample comprising a second plurality of nucleic acid molecules from the subject; processing, by a computer, the second plurality of nucleic acid molecules to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold. Additionally, in this aspect, the autosomal single nucleotide polymorphisms have a minor allele fraction that exceeds a pre-determined threshold. In some embodiments where the autosomal single nucleotide polymorphisms have a minor allele fraction that exceeds a particular threshold, the autosomal single nucleotide polymorphisms have a minor allele fraction that exceeds about 7.5%.

In some embodiments, the first plurality of nucleic acid molecules and the second plurality of nucleic acid molecules comprise cell-free DNA (cfDNA). In some embodiments, the first plurality of nucleic acid molecules and the second plurality of nucleic acid molecules comprise buffy coat DNA. In some embodiments, the first plurality of nucleic acid molecules and the second plurality of nucleic acid molecules comprise solid tumor DNA.

In some embodiments, the second biological sample is obtained from the subject at a later time after obtaining the first biological sample. In some embodiments, processing the first plurality of nucleic acid molecules comprises sequencing the first plurality of nucleic acid molecules to generate a first plurality of sequencing reads, and processing the second plurality of nucleic acid molecules comprises sequencing the second plurality of nucleic acid molecules to generate a second plurality of sequencing reads.

In some embodiments, the sequencing comprises whole genome sequencing (WGS). In some embodiments, the sequencing is performed at a depth of no more than about 10×. In some embodiments, the sequencing is performed at a depth of no more than about 8×. In some embodiments, the sequencing is performed at a depth of no more than about 6×. In some embodiments, the quantitative measure of the first plurality of nucleic acid molecules comprises a coverage of the first plurality of nucleic acid molecules at each of the plurality of genetic loci, and the quantitative measure of the second plurality of nucleic acid molecules comprises a coverage of the second plurality of nucleic acid molecules at each of the plurality of genetic loci.

In some embodiments, processing the first plurality of nucleic acid molecules comprises performing binding measurements of the first plurality of nucleic acid molecules, and processing the second plurality of nucleic acid molecules comprises performing binding measurements of the second plurality of nucleic acid molecules. In some embodiments, the quantitative measure of the first plurality of nucleic acid molecules at each of the plurality of genetic loci comprises a number of the first plurality of nucleic acid molecules containing the genetic locus, and the quantitative measure of the second plurality of nucleic acid molecules at each of the plurality of genetic loci comprises a number of the second plurality of nucleic acid molecules containing the genetic locus.

In some embodiments, the method further comprises enriching the first plurality of nucleic acid molecules and/or the second plurality of nucleic acid molecules for at least a portion of the plurality of genetic loci. In some embodiments, the enrichment comprises amplifying at least a portion of the first plurality of nucleic acid molecules and/or the second plurality of nucleic acid molecules. In some embodiments, the amplification comprises selective amplification. In some embodiments, the amplification comprises universal amplification. In some embodiments, the enrichment comprises selectively isolating at least a portion of the first plurality of nucleic acid molecules and/or the second plurality of nucleic acid molecules.

In some embodiments, the plurality of genetic loci comprises at least about 50 distinct autosomal single nucleotide polymorphisms (SNPs). In some embodiments, the plurality of genetic loci comprises at least about 100 distinct autosomal single nucleotide polymorphisms (SNPs).

In some embodiments, generating the first sample fingerprint further comprises obtaining a third biological sample comprising a third plurality of nucleic acid molecules from the subject, and processing the third plurality of nucleic acid molecules to obtain a quantitative measure of the third plurality of nucleic acid molecules at each of a second plurality of genetic loci, wherein the second plurality of genetic loci comprises autosomal single nucleotide polymorphisms (SNPs); and generating the second sample fingerprint further comprises obtaining a fourth biological sample comprising a fourth plurality of nucleic acid molecules from the subject, and processing the fourth plurality of nucleic acid molecules to obtain a quantitative measure of the fourth plurality of nucleic acid molecules at each of the second plurality of genetic loci.

In some embodiments, the third plurality of nucleic acid molecules and the fourth plurality of nucleic acid molecules comprise cell-free DNA (cfDNA). In some embodiments, the third plurality of nucleic acid molecules and the fourth plurality of nucleic acid molecules comprise buffy coat DNA. In some embodiments, the third plurality of nucleic acid molecules and the fourth plurality of nucleic acid molecules comprise solid tumor DNA. In some embodiments, generating the first sample fingerprint further comprises obtaining a fifth biological sample comprising a fifth plurality of nucleic acid molecules from the subject, and processing the fifth plurality of nucleic acid molecules to obtain a quantitative measure of the fifth plurality of nucleic acid molecules at each of a third plurality of genetic loci, wherein the third plurality of genetic loci comprises autosomal single nucleotide polymorphisms (SNPs); and generating the second sample fingerprint further comprises obtaining a sixth biological sample comprising a sixth plurality of nucleic acid molecules from the subject, and processing the sixth plurality of nucleic acid molecules to obtain a quantitative measure of the sixth plurality of nucleic acid molecules at each of the third plurality of genetic loci.

In some embodiments, the third plurality of nucleic acid molecules and the fourth plurality of nucleic acid molecules comprise cell-free DNA (cfDNA). In some embodiments, the third plurality of nucleic acid molecules and the fourth plurality of nucleic acid molecules comprise buffy coat DNA. In some embodiments, the third plurality of nucleic acid molecules and the fourth plurality of nucleic acid molecules comprise solid tumor DNA.

In some embodiments, the method comprises identifying the sample mismatch with a sensitivity of at least about 90%. In some embodiments, identifying the sample mismatch is performed with a sensitivity of at least about 95%. In some embodiments, the method comprises identifying the sample mismatch with a sensitivity of at least about 99%.

In some embodiments, the method comprises identifying the sample mismatch with a specificity of at least about 90%. In some embodiments, the method comprises identifying the sample mismatch with a specificity of at least about 95%. In some embodiments, the method comprises identifying the sample mismatch with a specificity of at least about 99%.

In some embodiments, the method comprises identifying the sample mismatch with a positive predictive value (PPV) of at least about 90%. In some embodiments, the method comprises identifying the sample mismatch with a positive predictive value (PPV) of at least about 95%. In some embodiments, the method comprises identifying the sample mismatch with a positive predictive value (PPV) of at least about 99%.

In some embodiments, the method comprises identifying the sample mismatch with a negative predictive value (NPV) of at least about 90%. In some embodiments, the method comprises identifying the sample mismatch with a negative predictive value (NPV) of at least about 95%. In some embodiments, the method comprises identifying the sample mismatch with a negative predictive value (NPV) of at least about 99%.

In some embodiments, the method comprises identifying the sample mismatch with an area under the curve (AUC) of at least about 0.90. In some embodiments, the method comprises identifying the sample mismatch with an area under the curve (AUC) of at least about 0.95. In some embodiments, the method comprises identifying the sample mismatch with an area under the curve (AUC) of at least about 0.99.

In some embodiments, the predetermined criterion is that the difference comprises a difference in genotype similarity greater than a predetermined threshold. In some embodiments, the predetermined threshold is about 0.8.

In some embodiments, the method further comprises excluding the second biological sample from further assaying based on the identified sample mismatch.

In some embodiments, the method further comprises identifying a sample match when the difference between the first sample fingerprint and the second sample fingerprint does not satisfy the predetermined criterion.

In some embodiments, the method comprises identifying the sample match with a sensitivity of at least about 90%. In some embodiments, the method comprises identifying the sample match with a sensitivity of at least about 95%. In some embodiments, the method comprises identifying the sample match with a sensitivity of at least about 99%.

In some embodiments, the method comprises identifying the sample match with a specificity of at least about 90%. In some embodiments, the method comprises identifying the sample match with a specificity of at least about 95%. In some embodiments, the method comprises identifying the sample match with a specificity of at least about 99%.

In some embodiments, the method comprises identifying the sample match with a positive predictive value (PPV) of at least about 90%. In some embodiments, the method comprises identifying the sample match with a positive predictive value (PPV) of at least about 95%. In some embodiments, the method comprises identifying the sample match with a positive predictive value (PPV) of at least about 99%.

In some embodiments, the method comprises identifying the sample match with a negative predictive value (NPV) of at least about 90%. In some embodiments, the method comprises identifying the sample match with a negative predictive value (NPV) of at least about 95%. In some embodiments, the method comprises identifying the sample match with a negative predictive value (NPV) of at least about 99%.

In some embodiments, the method comprises identifying the sample match with an area under the curve (AUC) of at least about 0.90. In some embodiments, the method comprises identifying the sample match with an area under the curve (AUC) of at least about 0.95. In some embodiments, the method comprises identifying the sample match with an area under the curve (AUC) of at least about 0.99.

In some embodiments, the method further comprises subjecting the second biological sample to further assaying based on the identified sample match. In some embodiments, the method further comprises, based on the identified sample match, storing the second sample fingerprint in a database, and optionally, storing the first sample fingerprint in the database.

In another aspect, the present disclosure provides a non-transitory computer-readable medium comprising machine-executable code that, upon execution by one or more computer processors, implements a method for identifying a sample mismatch, comprising: receiving information of a first sample fingerprint comprising a quantitative measure of a first plurality of nucleic acid molecules of a first biological sample at each of a plurality of genetic loci, wherein the plurality of genetic loci comprises autosomal single nucleotide polymorphisms (SNPs), and wherein the quantitative measure of the first plurality of nucleic acid molecules comprises no more than twelve independent measures of the plurality of nucleic acid molecules; receiving information of a second sample fingerprint comprising a quantitative measure of a second plurality of nucleic acid molecules of a second biological sample at each of the plurality of genetic loci, wherein the second biological sample is obtained from the subject; determining a difference between the first sample fingerprint and the second sample fingerprint; and identifying the sample mismatch when the difference between the first sample fingerprint and the second sample fingerprint satisfies a predetermined criterion.

In another aspect, the present disclosure provides a computer-implemented method for identifying a sample mismatch, comprising: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the quantitative measure of the first plurality of nucleic acid molecules comprises no more than twelve independent measures of the first plurality of nucleic acid molecules.

In another aspect, the present disclosure provides a computer-implemented method for identifying a sample mismatch, comprising: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the autosomal single nucleotide polymorphisms comprise simple single nucleotide polymorphisms.

In another aspect, the present disclosure provides a computer-implemented method for identifying a sample mismatch, comprising: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the autosomal single nucleotide polymorphisms have a minor allele fraction that exceeds a pre-determined threshold.

In another aspect, the present disclosure provides a system, comprising a controller comprising, or capable of accessing, computer readable media comprising non-transitory computer-executable instructions which, when executed by at least one electronic processor perform at least: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the quantitative measure of the first plurality of nucleic acid molecules comprises no more than twelve independent measures of the first plurality of nucleic acid molecules.

In another aspect, the present disclosure provides a system, comprising a controller comprising, or capable of accessing, computer readable media comprising non-transitory computer-executable instructions which, when executed by at least one electronic processor perform at least: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the autosomal single nucleotide polymorphisms comprise simple single nucleotide polymorphisms.

In another aspect, the present disclosure provides a system, comprising a controller comprising, or capable of accessing, computer readable media comprising non-transitory computer-executable instructions which, when executed by at least one electronic processor perform at least: processing a first plurality of nucleic acid molecules (e.g., from a first biological sample obtained from a subject) to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); processing the second plurality of nucleic acid molecules (e.g., from a second biological sample obtained from the subject) to generate a second sample fingerprint comprising a quantitative measure of the second plurality of nucleic acid molecules at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the autosomal single nucleotide polymorphisms have a minor allele fraction that exceeds a pre-determined threshold.

In another aspect, the present disclosure provides a computer-implemented method for identifying a sample mismatch, comprising: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the quantitative measure of the first plurality of nucleic acid molecules comprises no more than twelve independent measures of the first plurality of nucleic acid molecules.

In another aspect, the present disclosure provides a computer-implemented method for identifying a sample mismatch, comprising: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the autosomal single nucleotide polymorphisms comprise simple single nucleotide polymorphisms.

In another aspect, the present disclosure provides a computer-implemented method for identifying a sample mismatch, comprising: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying the sample mismatch when the difference between the first sample fingerprint and the second sample fingerprint exceeds a pre-determined threshold, wherein the autosomal single nucleotide polymorphisms have a minor allele fraction that exceeds a pre-determined threshold.

In another aspect, the present disclosure provides a system, comprising a controller comprising, or capable of accessing, computer readable media comprising non-transitory computer-executable instructions which, when executed by at least one electronic processor perform at least: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the quantitative measure of the first plurality of nucleic acid molecules comprises no more than twelve independent measures of the first plurality of nucleic acid molecules.

In another aspect, the present disclosure provides a system, comprising a controller comprising, or capable of accessing, computer readable media comprising non-transitory computer-executable instructions which, when executed by at least one electronic processor perform at least: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the autosomal single nucleotide polymorphisms comprise simple single nucleotide polymorphisms.

In another aspect, the present disclosure provides a system, comprising a controller comprising, or capable of accessing, computer readable media comprising non-transitory computer-executable instructions which, when executed by at least one electronic processor perform at least: obtaining a first sample fingerprint comprising a quantitative measure of a first plurality of nucleic acid molecules (e.g., from a first biological sample obtained from a subject) at each of a plurality of genetic loci, wherein the plurality of genetic loci comprises autosomal single nucleotide polymorphisms (SNPs); obtaining a second sample fingerprint comprising a quantitative measure of a second plurality of nucleic acid molecules (e.g., from a second biological sample obtained from the subject) at each of the plurality of genetic loci; determining a difference between the first sample fingerprint and the second sample fingerprint; and identifying a sample mismatch when the difference between the first sample fingerprint and the second sample fingerprint exceeds a pre-determined threshold, wherein the autosomal single nucleotide polymorphisms have a minor allele fraction that exceeds a pre-determined threshold.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a method for fingerprinting of biological samples, in accordance with some embodiments.

FIG. 2 illustrates an example of a method for identifying sample mismatches based on fingerprinting a first biological sample and a second biological sample, in accordance with some embodiments.

FIG. 3 illustrates a full visualization of comparisons of sample fingerprints generated from a plurality of assayed biological samples. The strong dark line along the diagonal indicates all samples that were not swapped (e.g., sample matches). The off-diagonal elements indicate samples that are too similar to samples that are supposed to have been obtained from a different subject (e.g., potential sample mismatches).

FIG. 4 illustrates an example of a clear internal sample mismatch (e.g., sample swap), in which a visualization of a comparison of assays performed on a large number of biological samples obtained from two different subjects. The off-diagonal bars next to the “broken” squares on the diagonal indicate that these two samples have been switched (BLIB00366 and BLIB00367).

FIG. 5 illustrates an image of a clear sample mismatch (e.g., sample swap) and an example of a sample discrepancy that cannot be resolved. The tissue samples obtained from a first patient (ID #4181) and a second patient (ID #4175) were swapped. One of the cfDNA samples for a third patient (ID #4161) does not match any other sample, including other samples that are supposed to be from the third patient (ID #4161). This sample was therefore excluded from further assays and processing.

FIG. 6 illustrates a plot showing the expected genotype similarities between pairs of samples from the same or different subjects (e.g., patients or persons). This plot illustrates how a suitable threshold is identified for distinguishing or differentiating between samples obtained from the same person versus samples obtained from different persons. After potential sample mismatches are accounted for by excluding samples suspected of being swapped and samples with low coverage (leading to a low number of genotype comparisons), the distributions are completely separated. Thus, thresholding can be performed at a genotype similarity of 0.8.

FIG. 7 illustrates a comparison of gender calls for a plurality of assayed DNA samples. X reads are shown on the X axis, and Y reads are shown on the Y axis. The blue samples are supposed to have been obtained from male subjects, the red samples are supposed to have been obtained from female subjects, and the gray samples had such information unavailable. A first set of data points located well above the threshold line are called as male, and a second set of data points located well below the threshold line are called as female. The plot shows a few blue data points located below the threshold line and a few red data points located above the threshold, which correspond to samples which are identified as sample mismatches (e.g., that are identified as being swapped). The data points that fall right on the threshold line were obtained from a cancer patient with a large portion of chromosome X duplicated.

FIG. 8 illustrates a computer system that is programmed or otherwise configured to implement methods provided herein.

### DETAILED DESCRIPTION

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups, individually or in combination.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double stranded. A nucleic acid molecule may be linear, curved, or circular or any combination thereof.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb or it may have any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. The nucleic acid molecules may be cell-free or cell-free nucleic acid molecules, such as cell-free DNA (cfDNA) or cell-free RNA (cfRNA). The nucleic acid molecules may be buffy coat nucleic acid molecules, such as buffy coat DNA. The nucleic acid molecules may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides (e.g., cfDNA) may be fetal in origin (via fluid taken from a pregnant subject), or may be derived from tissue of the subject itself.

The term “subject,” as used herein, generally refers to an individual having a biological sample that is undergoing processing or analysis. A subject can be an animal or plant. The subject can be a mammal, such as a human, dog, cat, horse, pig or rodent. The subject can be a patient, e.g., have or be suspected of having a disease, such as one or more cancers, one or more infectious diseases, one or more genetic disorder, or one or more tumors, or any combination thereof. For subjects having or suspected of having one or more tumors, the tumors may be of one or more types.

The term “whole blood,” as used herein, generally refers to a blood sample that has not been separated into sub-components (e.g., by centrifugation). The whole blood of a blood sample may contain cfDNA and/or germline DNA. Whole blood DNA (which may contain cfDNA and/or germline DNA) may be extracted from a blood sample. Whole blood DNA sequencing reads (which may contain cfDNA sequencing reads and/or germline DNA sequencing reads) may be extracted from whole blood DNA.

The collection and assaying of biological samples obtained from subjects may often encounter challenges with reliable maintenance of sample identity throughout clinical and laboratory processes. For example, biological samples may often be inadvertently swapped in laboratory or clinical settings, thereby resulting in potentially incorrect clinical results if left undetected and uncorrected.

Methods for fingerprinting biological samples using panels of genetic loci may require sufficiently deep coverage to obtain genetic information at a desired sensitivity, specificity, or accuracy. For example, deep coverage may be required for sufficient signal-to-noise (SNR) ratio to distinguish between fingerprints generated from different samples. Such samples may be longitudinal samples, e.g., obtained from the same subject at two different time points. Longitudinal samples processed using low-pass sequencing may encounter challenges with (1) correcting matching together samples from different time points and (2) identifying a panel of genetic loci suitable for sample fingerprinting despite relative low read coverage at any one location.

Methods and systems are provided for generating and comparing fingerprints of biological samples. Sample fingerprints may be generated by sequencing one or more sets of nucleic acid molecules from biological samples obtained from a subject at each of one or more time points. Pairwise comparison of sample fingerprints may be performed to determine whether a sample mismatch (e.g., that the two samples were obtained from different subjects) or a sample match (e.g., that the two samples were obtained from the same subject) is present between the two biological samples from which the sample fingerprints were generated.

In an aspect, the present disclosure provides a method for generating a sample fingerprint, comprising: obtaining a biological sample comprising a plurality of nucleic acid molecules from a subject; and processing the plurality of nucleic acid molecules to generate a sample fingerprint comprising a quantitative measure of the plurality of nucleic acid molecules at each of a plurality of genetic loci, wherein the plurality of genetic loci comprises autosomal single nucleotide polymorphisms (SNPs). The generated sample fingerprint may be stored in a database.

In another aspect, the present disclosure provides a method for identifying a sample mismatch, comprising: obtaining a first biological sample comprising a first plurality of nucleic acid molecules from a subject; processing the first plurality of nucleic acid molecules to generate a first sample fingerprint comprising a quantitative measure of the first plurality of nucleic acid molecules at each of a plurality of genetic loci, wherein the plurality of genetic loci comprises autosomal single nucleotide polymorphisms (SNPs); obtaining a second biological sample comprising a second plurality of nucleic acid molecules from the subject; processing the second plurality of nucleic acid molecules to generate a second sample fingerprint comprising a quantitative measure of the second plurality of nucleic acid molecules at each of the plurality of genetic loci; determining a difference between the first sample fingerprint and the second sample fingerprint; and identifying the sample mismatch when the difference between the first sample fingerprint and the second sample fingerprint satisfies a predetermined criterion.

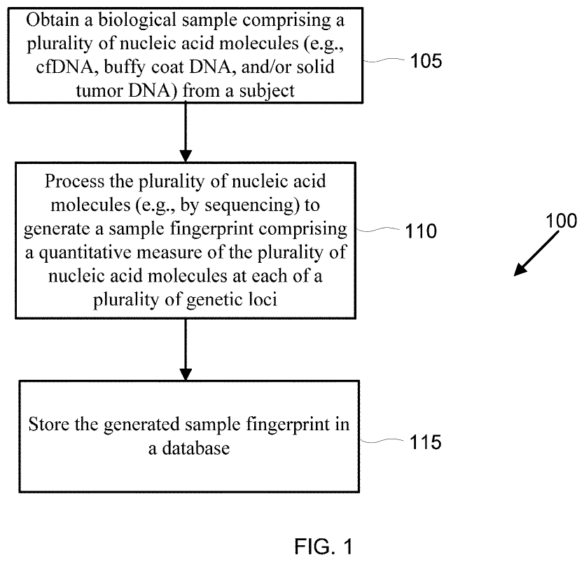

FIG. 1 illustrates an example of a method for generating a sample fingerprint of a biological sample, in accordance with some embodiments. The method for generating a sample fingerprint may comprise obtaining a biological sample comprising a plurality of nucleic acid molecules from a subject. In some embodiments, the plurality of nucleic acid molecules may comprise a plurality of cell-free DNA (cfDNA) molecules, a plurality of buffy coat DNA molecules, a plurality of solid tumor DNA molecules, or a combination thereof (as in operation 105).

The method for generating a sample fingerprint may comprise processing the plurality of nucleic acid molecules to generate a sample fingerprint comprising a quantitative measure of the plurality of nucleic acid molecules at each of a plurality of genetic loci. In some embodiments, processing the plurality of nucleic acid molecules comprises sequencing the plurality of nucleic acid molecules to generate sequencing reads at each of the plurality of genetic loci (as in operation 110).

In some embodiments, the plurality of genetic loci may comprise a plurality of distinct autosomal SNPs. In some examples, the plurality of genetic loci that are analyzed may comprise more than about 100 genetic loci. In some examples, the plurality of genetic loci that are analyzed may comprise more than about 200 genetic loci, more than about 300 genetic loci, more than about 500 genetic loci, more than about 1,000 genetic loci, more than about 1,500 genetic loci, more than about 2,000 genetic loci, more than about 2,500 genetic loci, more than about 3,000 genetic loci, more than about 3,500 genetic loci, more than about 4,000 genetic loci, more than about 4,500 genetic loci, more than about 5,000 genetic loci, or more than about 5,500 genetic loci. In some examples, a genetic locus having a distinct autosomal SNP may include rs2839, an annotated SNP located on chromosome 1 which is included in public databases such as dbSNP. In some examples, distinct autosomal SNPs, such as rs2839, suitable for use as part of a sample fingerprint profile may be identified by, for example, filtering databases of known SNPs based on quality criteria or analyzing large data sets of genomic data from a large set of human participants to call SNPs which meet quality and reliability standards.

In some embodiments, SNPs may be filtered for certain criteria, such as those SNPs that can uniquely identify a personal genome. Such a set of SNPs may collectively provide an extremely small likelihood that two individuals have the same genomic profile (e.g., for a sample fingerprint). For example, SNPs with reported allele frequencies across five major continental populations (e.g., from the 1000 genomes project and the ExAC Consortium) may serve as candidate SNPs to be further analyzed for inclusion in a sample fingerprint profile. As another example, SNPs that may be used to predict ABO blood type of a subject may be used. As another example, SNPs that may be used to predict sex of a subject may be used. Methods of selecting SNPs may be as described by, for example, Du et al. (“A SNP panel and online tool for checking genotype concordance through comparing QR codes”, PLOS One, 2017) and Hu et al. (“Evaluating information content of SNPs for sample-tagging in re-sequencing projects”, Scientific Reports, 2015), each of which is hereby incorporated by reference in its entirety.

In some examples, SNPs may be filtered to select autosomal SNPs. In some examples, SNPs may be filtered to select simple SNPs. Simple SNPs may comprise SNPs that have only two alleles that have no insertions or deletions. Simple SNPs may have only a single base change. In some examples, SNPs may be annotated in the dbSNP with a low reference SNP ID (rs number). These rs numbers are assigned sequentially at the time of the submission to the database. In some cases, earlier submissions having lower rs numbers may have fewer technical artifacts. In some examples, SNPs may be filtered to have a minor allele fraction greater than a certain threshold. In some examples, SNPs may be filtered to have a minor allele fraction greater than about 1%, greater than about 1.5%, greater than about 2%, greater than about 2.5%, greater than about 3%, greater than about 3.5%, greater than about 4%, greater than about 4.5%, greater than about 5%, greater than about 5.5%, greater than about 6%, greater than about 6.5%, greater than about 7%, greater than about 7.5%, greater than about 8%, greater than 8.5%, greater than about 9%, greater than about 9.5%, or greater than about 10%.

In some embodiments, the method for generating a sample fingerprint may further comprise storing the generated sample fingerprint in a database (as in operation 115).

For example, sequencing reads may be generated from the nucleic acid molecules using any suitable sequencing method. The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms.

In some embodiments, the sequencing comprises whole genome sequencing (WGS). The sequencing may be performed at a depth sufficient to generate a sample fingerprint from a biological sample obtained from a subject or to identify a sample mismatch or a sample match based on a difference between two sample fingerprints with a desired performance (e.g., accuracy, sensitivity, specificity, positive predictive value (PPV), negative predictive value (NPV), or the area under curve (AUC) of a receiver operator characteristic (ROC)). In some embodiments, the sequencing is performed in a “low-pass” manner, for example, at a depth of no more than about 12×, no more than about 11×, no more than about 10×, no more than about 9×, no more than about 8×, no more than about 7×, no more than about 6×, no more than about 5×, no more than about 4×, no more than about 3×, no more than about 2×, or no more than about 1×.

In some embodiments, generating a sample fingerprint from a biological sample obtained from a subject may comprise aligning the sequencing reads to a reference genome. The reference genome may comprise at least a portion of a genome (e.g., the human genome). The reference genome may comprise an entire genome (e.g., the entire human genome). The reference genome may comprise a database comprising a plurality of genomic regions that correspond to coding and/or non-coding genomic regions of a genome. The database may comprise a plurality of genomic regions that correspond to coding and/or non-coding genomic regions of a genome, such as single nucleotide polymorphisms (SNPs), single nucleotide variants (SNVs), copy number variants (CNVs), insertions or deletions (indels), fusion genes, and repeat elements. The alignment may be performed using a Burrows-Wheeler algorithm or other alignment algorithms.

In some embodiments, generating a sample fingerprint from a biological sample obtained from a subject may comprise generating a quantitative measure of the sequencing reads for each of a plurality of genetic loci. Quantitative measures of the sequencing reads may be generated, such as counts of sequencing reads that are aligned with a given genetic locus.

In some embodiments, the method for generating a sample fingerprint from a biological sample obtained from a subject may comprise generating base calls (e.g., including uncertain calls for some bases) at each of a plurality of SNPs for each of one or more DNA samples (e.g., cfDNA, buffy coat DNA, and/or solid tumor DNA). Base calls may be generated, for example, using GATK or other SNP calling packages.

In some embodiments, the generated sample fingerprint from the biological sample obtained from the subject may be stored in a database to represent a set of one or more biological samples obtained from the subject. The set of biological samples may represent one or more types of DNA samples (e.g., cfDNA, buffy coat DNA, and/or solid tumor DNA) collected at one or more time points. A sample fingerprint stored in the database may have a data size of no more than about 1 gigabyte (GB), no more than about 500 megabytes (MB), no more than about 100 MB, no more than about 50 MB, no more than about 10 MB, no more than about 5 MB, no more than about 1 MB, no more than about 500 kilobytes (KB), no more than about 250 KB, or no more than about 100 KB.

In some embodiments, the plurality of SNPs may be a very large set of well-behaved SNPs spread across the genome. Each of the SNPs may provide some information content which may not be very high. The plurality of SNPs may be autosomal SNPs. The plurality of SNPs may be located not in close proximity to telomeres. The plurality of SNPs may be annotated in dbSNP with an ID indicating generation before a certain date. The plurality of SNPs may have a minor allele fraction (MAF) greater than about 1%, with only two alleles. In some embodiments, the plurality of SNPs may have a minor allele fraction (MAF) greater than about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 169%, 16.5%, 17%, 17.5%, 18%, 18.5%, 19%, 19.5%, 20%, 20.5%, 21%, 21.5%), 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, or greater than 45%, with only two alleles.

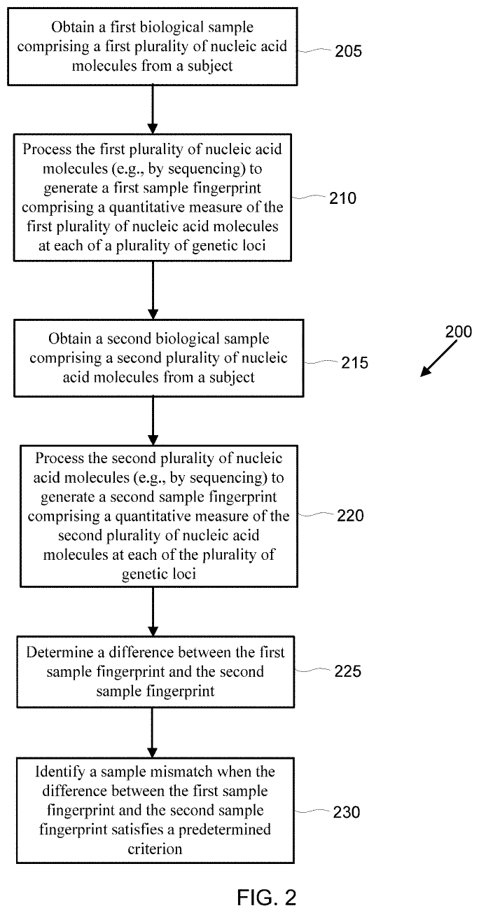

FIG. 2 illustrates an example of a method for identifying sample mismatches based on fingerprinting a first biological sample and a second biological sample, in accordance with some embodiments. In some embodiments, the method for generating sample fingerprints from biological samples obtained from a subject may comprise collecting cell-free DNA (cfDNA) samples, buffy coat DNA samples, and/or solid tumor DNA samples at a baseline time point and at one or more subsequent time points. Each set of DNA samples obtained from the subject at or around the same baseline time point may be processed to generate a baseline sample fingerprint for the subject corresponding to the baseline time point. Each set of DNA samples obtained from the subject at or around the same subsequent time point may be processed to generate a subsequent sample fingerprint for the subject corresponding to the subsequent time point.

For example, a first biological sample comprising a first plurality of nucleic acid molecules may be obtained from a subject (as in operation 205). The first plurality of nucleic acid molecules may be processed to generate a first sample fingerprint comprising a quantitative measure of the first plurality at each of a plurality of genetic loci (as in operation 210). In some embodiments, the plurality of genetic loci comprises autosomal single nucleotide polymorphisms (SNPs). Next, a second biological sample comprising a second plurality of nucleic acid molecules may be obtained from the subject (as in operation 215). The second plurality of nucleic acid molecules may be processed to generate a second sample fingerprint comprising a quantitative measure of the second plurality at each of the plurality of genetic loci (as in operation 220). Next, a difference between the first sample fingerprint and the second sample fingerprint may be determined (as in operation 225). Next, the sample mismatch may be identified when the difference satisfies a predetermined criterion (as in operation 230).

In some embodiments, after a plurality of sample fingerprints are generated from biological samples obtained from a subject, the sample fingerprints may be processed to generate pairwise comparisons of the sequence data of the sample fingerprints. The pairwise comparisons of the sequence data of the sample fingerprints may be performed to ensure that (a) all pairs of samples that are supposed to be from the same subject (person) are indeed from the same subject (person), (b) all pairs of samples that are supposed to be from different subjects (people) are indeed from different subjects (people), and (c) all samples have X and Y chromosome reads in accordance with the expectation from the sex of the subject from which the samples are obtained. For example, pairwise comparisons between two samples may be performed by comparing the first sample's fingerprint (using quantitative measures obtained by assaying cfDNA, buffy coat DNA, and/or solid tumor DNA) with the second sample's fingerprint (using quantitative measures obtained by assaying the same types of DNA available in the first sample fingerprint). For example, such quantitative measures may be generated by sequencing the nucleic acid molecules or by performing binding measurements of the nucleic acid molecules.

Performing pairwise comparisons of the sequence data of the sample fingerprints may comprise generating a quantitative measure of genotype similarity, by comparing each of the SNP calls in which a sufficient number of reads in both samples is present in order to have a desired degree of confidence in the accuracy of the call. For a given SNP, a number of reads may be judged as sufficient when greater than a predetermined threshold for the given SNP. Such predetermined thresholds may be identified for each SNP based on analysis of patient data (e.g., for patients with known SNP status). For example, the predetermined threshold for each SNP may be determined based on taking into account a lower number of reads needed to make a confident call for a heterozygous call than a homozygous call.

Performing pairwise comparisons of the sequence data of the sample fingerprints may comprise identifying two samples as being from the same subject (person) (e.g., a sample match) or not being from the same subject (person) (e.g., a sample mismatch) based at least in part on the fraction of genotype calls that are identical between the two sample fingerprints. For example, the fraction of genotype calls that are identical between the two sample fingerprints may be compared to a predetermined threshold to identify a sample mismatch or a sample match. The predetermined threshold may be generated by analyzing a large amount of data aggregated from a large number of sample fingerprints generated from a plurality of subjects, and selecting the predetermined threshold that optimizes a desired performance (e.g., accuracy, sensitivity, specificity, positive predictive value (PPV), negative predictive value (NPV), or the area under curve (AUC) of a receiver operator characteristic (ROC)).

Performing pairwise comparisons of the sequence data of the sample fingerprints may comprise generating a heatmap of the genotype similarities for all pairs of samples, grouped by subject (person). In these visualizations, internal sample swaps (e.g., sample mismatches occurring in a laboratory setting of a user) may be revealed as dark squares off the diagonal coupled with light squares on the edge of the diagonal. External sample swaps (e.g., sample mismatches occurring at the clinic or other sample collection site) may be revealed as light “gaps” in the on-diagonal squares. To aid in this visualization, generation of the heatmap may be limited to a set of samples that are suspected to be swapped.

Performing pairwise comparisons of the sequence data of the sample fingerprints may comprise comparison of X and Y chromosome reads. For example, comparison of X and Y chromosome reads may be performed to detect sample swaps (sample mismatches) between samples of different sex. A ratio of Y reads (e.g., sequence reads mapping to a Y sex chromosome) to X reads (e.g., sequence reads mapping to an X sex chromosome) may be determined. The ratio of Y reads to X reads (Y/X read ratio) may be compared to known distributions of Y/X ratios present in male subjects and female subjects. Each sample may be classified as male or female or ambiguous, based on the generated Y/X read ratio.

The sex classification of the sample may be compared to the subject's known sex to determine a performance metric (e.g., sensitivity, specificity, positive predictive value, negative predictive value, or area-under-the-curve) of the sex classification. For example, ambiguous classifications may be generated from analyzing samples where a tumor has amplified part of the chromosome X in a male, thereby resulting in Y/X read ratios much lower than those in the unaffected male population. If a sample's sex classification does not match the subject's (patient's) known sex, then the sample is specifically suspected of being swapped. Such results may be fed into and disambiguate the method for sex classification of samples and provide an indication of where the swap occurred (e.g., laboratory setting or clinical setting).

The identification information of swapped samples (e.g., sample mismatches or sample matches) and the identification information of sex mismatch based on analyzing the X and Y chromosomes may be compared to a database containing records of proximate samples (e.g., samples which were next to each other at certain steps in sample processing) to reveal the exact circumstances under which the detected sample swap has occurred. In many cases, such comparisons allow correction of the identified sample mismatch by reassigning sample identification information to their correct subjects. In some cases, correction of the identified sample mismatch may not be possible, such as if, for example, a sample fingerprint does not match any other samples that have been assayed. Such cases may be caused by being sent the wrong sample from an external partner or a sample swap with a sample that has yet to be assayed. In such cases, such indeterminate samples can be marked in the database and excluded from further analyses.

In some embodiments, processing the first plurality of nucleic acid molecules comprises performing binding measurements of the first plurality of nucleic acid molecules, and processing the second plurality of nucleic acid molecules comprises performing binding measurements of the second plurality of nucleic acid molecules. In some embodiments, the quantitative measure of the first plurality of nucleic acid molecules at each of the plurality of genetic loci comprises a number of the first plurality of nucleic acid molecules containing the genetic locus, and the quantitative measure of the second plurality of nucleic acid molecules at each of the plurality of genetic loci comprises a number of the second plurality of nucleic acid molecules containing the genetic locus. For example, the binding measurements may be obtained by assaying the plurality of nucleic acid molecules using probes that are selective for at least a portion of the plurality of SNPs in the plurality of nucleic acid molecules. In some embodiments, the probes are nucleic acid molecules having sequence complementarity with nucleic acid sequences of the plurality of SNPs. In some embodiments, the probes are nucleic acid molecules which are primers or enrichment sequences. In some embodiments, the assaying comprises use of array hybridization or polymerase chain reaction (PCR), or nucleic acid sequencing.

In some embodiments, the method further comprises enriching the plurality of nucleic acid molecules for at least a portion of the plurality of SNPs. In some embodiments, the enrichment comprises amplifying the plurality of nucleic acid molecules. For example, the plurality of nucleic acid molecules may be amplified by selective amplification (e.g., by using a set of primers or probes comprising nucleic acid molecules having sequence complementarity with nucleic acid sequences of the plurality of SNPs). Alternatively or in combination, the plurality of nucleic acid molecules may be amplified by universal amplification (e.g., by using universal primers). In some embodiments, the enrichment comprises selectively isolating at least a portion of the plurality of nucleic acid molecules.

The plurality of genetic loci may comprise at least about 10 distinct autosomal single nucleotide polymorphisms (SNPs), at least about 50 distinct autosomal SNPs, at least about 100 distinct autosomal SNPs, at least about 500 distinct autosomal SNPs, at least about 1 thousand distinct autosomal SNPs, at least about 5 thousand distinct autosomal SNPs, at least about 10 thousand distinct autosomal SNPs, at least about 50 thousand distinct autosomal SNPs, at least about 100 thousand distinct autosomal SNPs, at least about 500 thousand distinct autosomal SNPs, at least about 1 million distinct autosomal SNPs, at least about 2 million distinct autosomal SNPs, at least about 3 million distinct autosomal SNPs, at least about 4 million distinct autosomal SNPs, at least about 5 million distinct autosomal SNPs, at least about 10 million distinct autosomal SNPs, or more than about 10 million distinct autosomal SNPs.

In some embodiments, identifying the sample mismatch is performed with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The sensitivity of identifying a sample mismatch may be measured or estimated as the percentage of sample mismatches that are expected to be identified using a method of the present disclosure. The sensitivity may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying the sample mismatch is performed with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99% at least about 99.5%, at least about 99.6%, at least about 99.7%, at least about 99.8° 43, at least about 99.9%, at least about 99.99%, or at least about 99.999%. The specificity of identifying a sample mismatch may be measured or estimated as the percentage of samples that are not mismatches (e.g., sample matches) that are expected to be identified using a method of the present disclosure. The specificity may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying the sample mismatch is performed with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The PPV of identifying a sample mismatch may be measured or estimated as the likelihood that a sample mismatch identified using a method of the present disclosure is a true positive (e.g., that a pair of samples are truly mismatched with each other, given that the method has identified the pair of samples as a mismatch). The PPV may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying the sample mismatch is performed with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The NPV of identifying a sample mismatch may be measured or estimated as the likelihood that a sample identified as not a mismatch (e.g., a sample match) using a method of the present disclosure is a true negative (e.g., that a pair of samples are truly not mismatched with each other, given that the method has identified the pair of samples as not a mismatch). The NPV may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying the sample mismatch is performed with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, at least about 0.99, at least about 0.995, at least about 0.996, at least about 0.997, at least about 0.998, at least about 0.999, at least about 0.9999, or at least about 0.99999.

In some embodiments, the method further comprises identifying a sample match when the difference between the first sample fingerprint and the second sample fingerprint does not satisfy the predetermined criterion.

In some embodiments, identifying a sample match is performed with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The sensitivity of identifying a sample match may be measured or estimated as the percentage of sample matches that are expected to be identified using a method of the present disclosure. The sensitivity may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying a sample match is performed with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The specificity of identifying a sample match may be measured or estimated as the percentage of samples that are not matches (e.g., sample mismatches) that are expected to be identified using a method of the present disclosure. The specificity may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying a sample match is performed with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The PPV of identifying a sample match may be measured or estimated as the likelihood that a sample match identified using a method of the present disclosure is a true positive (e.g., that a pair of samples are truly matched with each other, given that the method has identified the pair of samples as a match). The PPV may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying a sample match is performed with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.99%, or at least about 99.999%. The NPV of identifying a sample match may be measured or estimated as the likelihood that a sample identified as not a match (e.g., a sample mismatch) using a method of the present disclosure is a true negative (e.g., that a pair of samples are truly not matched with each other, given that the method has identified the pair of samples as not a match). The NPV may be measured or estimated under assumptions of obtaining sufficient coverage across a certain number of distinct genetic loci (e.g., autosomal SNPs) and no sample quality issues (e.g., partial contamination such as sample mixing).

In some embodiments, identifying a sample match is performed with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, at least about 0.99, at least about 0.995, at least about 0.996, at least about 0.997, at least about 0.998, at least about 0.999, at least about 0.9999, or at least about 0.99999.

In some embodiments, the method of identifying a sample mismatch further comprises determining whether the difference between the first sample fingerprint and the second sample fingerprint satisfies a predetermined criterion. The predetermined threshold may be generated by generating sample fingerprints from one or more samples from one or more control subjects and identifying a suitable predetermined threshold based on the variability of the control samples (within the same subject and across different subjects (e.g., of different sex)).

The predetermined threshold may be adjusted based on a desired sensitivity, specificity, positive predictive value (PPV), negative predictive value (NPV), or accuracy of identifying a sample mismatch and/or a sample match. For example, the predetermined threshold may be adjusted to be lower if a high sensitivity of identifying a sample mismatch is desired. Alternatively, the predetermined threshold may be adjusted to be higher if a high specificity of identifying a sample mismatch is desired. The predetermined threshold may be adjusted so as to maximize the area under curve (AUC) of a receiver operator characteristic (ROC) of the control samples obtained from the control subjects. The predetermined threshold may be adjusted so as to achieve a desired balance between false positives (FPs) and false negatives (FNs) in identifying a sample mismatch and/or a sample match.

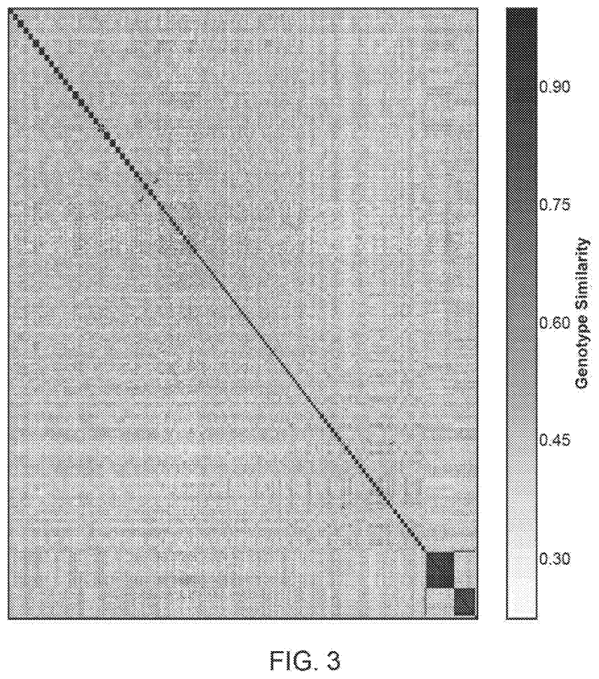

FIG. 3 illustrates a full visualization of comparisons of sample fingerprints generated from a plurality of assayed biological samples. The strong dark line along the diagonal indicates all samples that were not swapped (e.g., sample matches). For example, such sample matches may correspond to pairs of samples with matching patient identification information (e.g., ID number, date of birth, sex, etc.) being identified as truly belonging to the same patient. The off-diagonal elements indicate samples that are too similar to samples that are supposed to have been obtained from a different subject. For example, such sample mismatches may correspond to pairs of samples with matching patient identification information (e.g., ID number, date of birth, sex, etc.) being identified as likely to have been obtained from different patients (e.g., a potential sample swap). In the case of an identified sample mismatch, the mismatched sample fingerprint can be compared to other sample fingerprints (purportedly belonging to other patients) stored in the database with mismatching patient identification information (e.g., ID number, date of birth, sex, etc.) to attempt to identify and correct the sample mismatch. The sample mismatch can be corrected by swapping or updating the patient identification information associated with the sample fingerprints to match their correct identities, if found in the database. If the correct identity of a mismatched sample cannot be determined (e.g., if not found in the database), the mismatched sample can be marked for exclusion from further assays and processing.

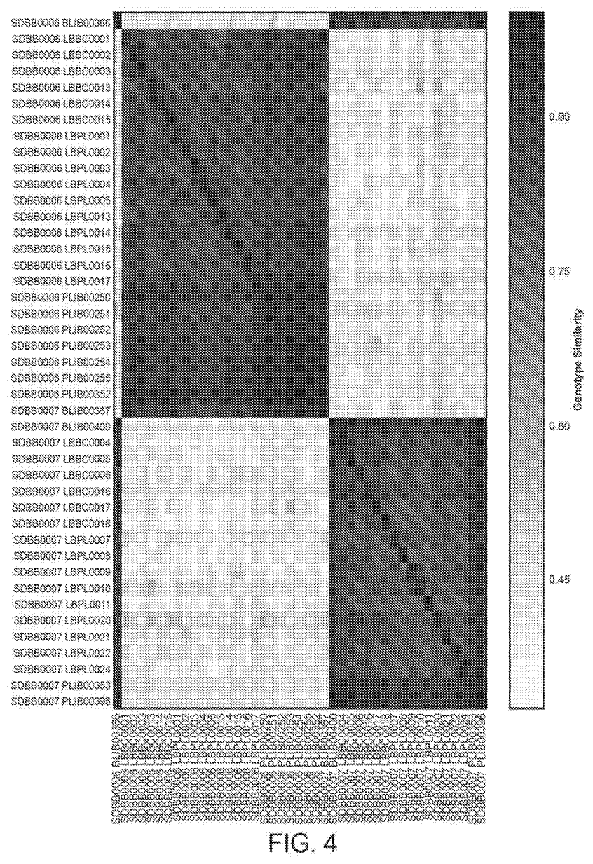

FIG. 4 illustrates an example of a clear internal sample mismatch (e.g., sample swap), in which a visualization of a comparison of assays performed on a large number of biological samples obtained from two different subjects. The off-diagonal bars next to the “broken” squares on the diagonal indicate that these two samples have been switched (BLIB00366 and BLIB00367). The sample mismatch can be corrected by swapping or updating the patient identification information associated with the pair of sample fingerprints to match their correct identities, since they were found in the database.

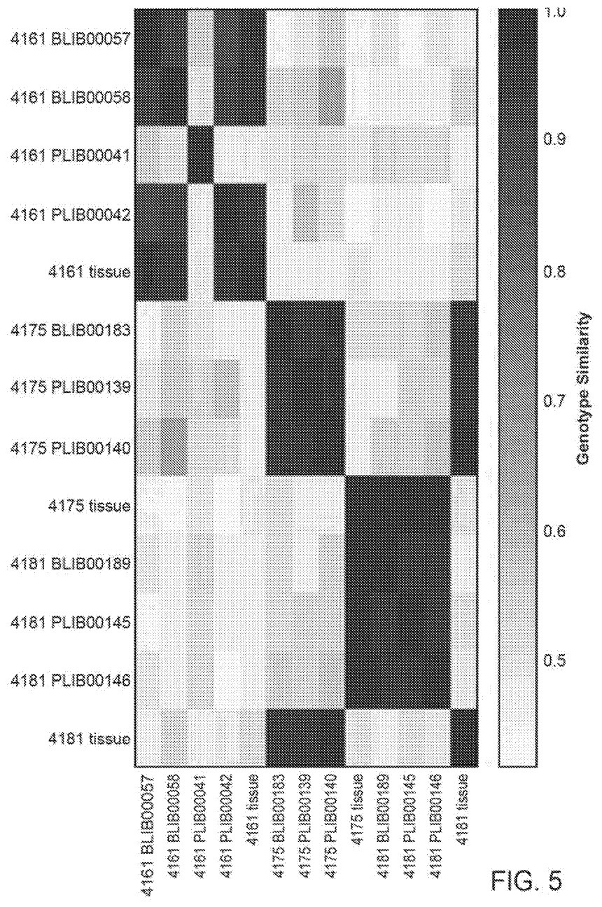

FIG. 5 illustrates an image of a clear sample mismatch (e.g., sample swap) and an example of a sample discrepancy that cannot be resolved. The tissue samples obtained from a first patient (ID #4181) and a second patient (ID #4175) were swapped. One of the cfDNA samples for a third patient (ID #4161) does not match any other sample, including other samples that are supposed to be from the third patient (ID #4161). Since the correct identity of the mismatched sample for the third patient (ID #4161) (having a sample discrepancy) cannot be determined (e.g., was not found in the database), the mismatched sample can be marked for exclusion from further assays and processing.

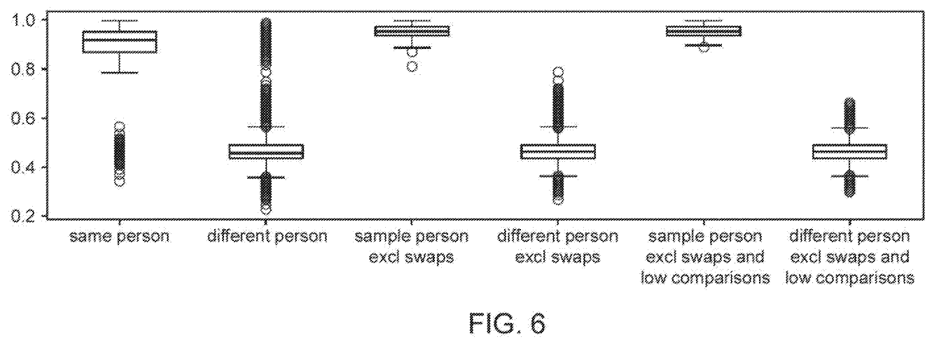

FIG. 6 illustrates a plot showing the expected genotype similarities between pairs of samples from the same or different subjects (e.g., patients or persons). This plot illustrates how a suitable threshold is identified for distinguishing or differentiating between samples obtained from the same person versus samples obtained from different persons. After potential sample mismatches are accounted for by excluding samples suspected of being swapped and samples with low coverage (leading to a low number of genotype comparisons), the distributions are completely separated.

For example, by excluding samples suspected of being swapped, the distribution of the expected genotype similarities between pairs of samples from the same person shifts upward (from the first column to the third column). By further excluding samples with low coverage (leading to a low number of genotype comparisons), the distribution of the expected genotype similarities between pairs of samples from the same person further shifts upward (from the third column to the fifth column). Similarly, by excluding samples suspected of being swapped, the distribution of the expected genotype similarities between pairs of samples from different persons shifts downward (from the second column to the fourth column). By further excluding samples with low coverage (leading to a low number of genotype comparisons), the distribution of the expected genotype similarities between pairs of samples from different persons further shifts downward (from the fourth column to the sixth column). Thus, in this example, thresholding between cases of samples from the same person (excluding swaps and low coverage) (fifth column) and cases of samples from different persons (excluding swaps and low coverage) (sixth column) can be accurately performed at a genotype similarity of 0.8. Since there is good separation between the similarity metrics of sample fingerprints obtained from the same subject as compared to sample fingerprints obtained from different subjects, a range of possible cutoff values (predetermined criteria) for genotype similarity may be used for accurately determining a sample match and/or a sample mismatch. The predetermined criterion may be set at a relatively high value to avoid or minimize the probability of false positive match calls, for example, when analyzing samples obtained from different but related subjects.

A predetermined criterion for determining a sample mismatch may be that a difference in genotype similarity between two sample fingerprints is greater than a predetermined threshold. Such a predetermined threshold may be, for example, a difference in genotype similarity of at least about 0.05, at least about 0.1, at least about 0.15, at least about 0.2, at least about 0.25, at least about 0.3, at least about 0.35, at least about 0.4, at least about 0.45, at least about 0.5, at least about 0.55, at least about 0.6, at least about 0.65, at least about 0.7, at least about 0.75, at least 0.8, at least about 0.85, or at least about 0.9.

Similarly, a predetermined criterion for determining a sample match may be that a difference in genotype similarity between two sample fingerprints is no more than a predetermined threshold. Such a predetermined threshold may be, for example, a difference in genotype similarity of no more than about 0.05, no more than about 0.1, no more than about 0.15, no more than about 0.2, no more than about 0.25, no more than about 0.3, no more than about 0.35, no more than about 0.4, no more than about 0.45, no more than about 0.5, no more than about 0.55, no more than about 0.6, no more than about 0.65, no more than about 0.7, no more than about 0.75, no more than 0.8, no more than about 0.85, or no more than about 0.9.

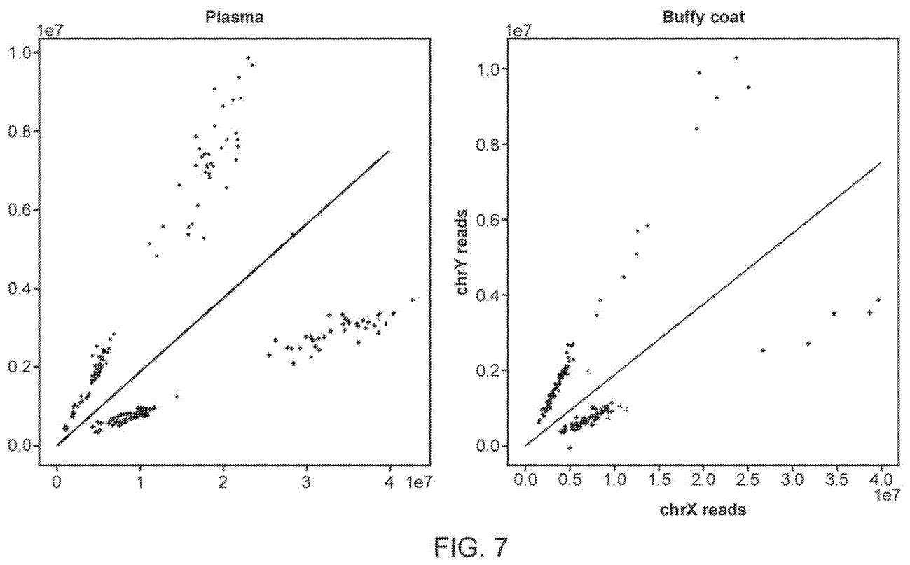

FIG. 7 illustrates a comparison of gender calls for a plurality of assayed DNA samples. X reads are shown on the X axis, and Y reads are shown on the Y axis. The blue samples are supposed to have been obtained from male subjects, the red samples are supposed to have been obtained from female subjects, and the gray samples had such information unavailable. A first set of data points located well above the threshold line are called as male, and a second set of data points located well below the threshold line are called as female. The plot shows a few blue data points located below the threshold line and a few red data points located above the threshold, which correspond to samples which are identified as sample mismatches (e.g., that are identified as being swapped). The data points that fall right on the threshold line were obtained from a cancer patient with a large portion of chromosome X duplicated.

### Computer Systems

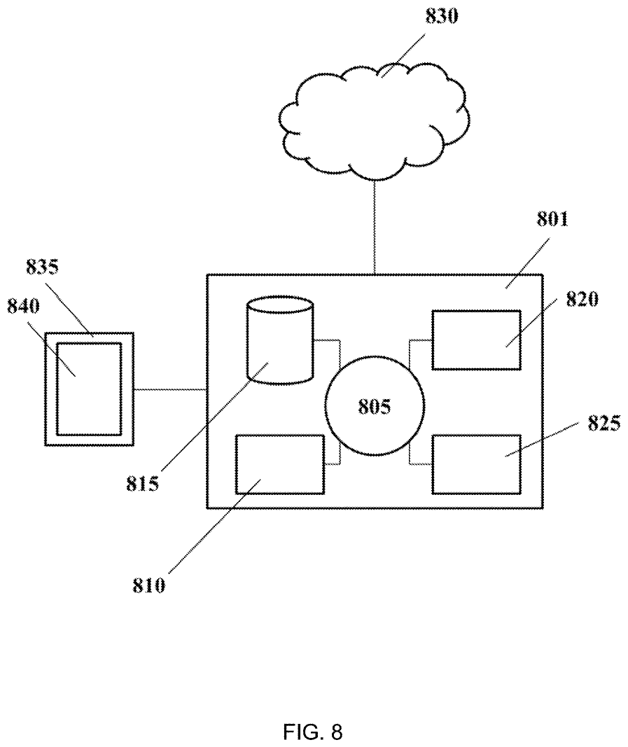

The present disclosure provides computer systems that are programmed to implement methods of the disclosure. FIG. 8 shows a computer system 801 that is programmed or otherwise configured to, for example, process nucleic acid molecules to generate a sample fingerprint comprising a quantitative measure of the nucleic acid molecules at each of a plurality of genetic loci, determine a difference between two sample fingerprints, and identify a sample mismatch when the difference between two sample fingerprints satisfies a predetermined criterion. The computer system 801 can regulate various aspects of analysis, calculation, and generation of the present disclosure, such as, for example, processing nucleic acid molecules to generate a sample fingerprint comprising a quantitative measure of the nucleic acid molecules at each of a plurality of genetic loci, determining a difference between two sample fingerprints, and identifying a sample mismatch when the difference between two sample fingerprints satisfies a predetermined criterion. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. For example, one or more computer servers may enable cloud computing over the network 830 (“the cloud”) to perform various aspects of analysis, calculation, and generation of the present disclosure, such as, for example, processing nucleic acid molecules to generate a sample fingerprint comprising a quantitative measure of the nucleic acid molecules at each of a plurality of genetic loci, determining a difference between two sample fingerprints, and identifying a sample mismatch when the difference between two sample fingerprints satisfies a predetermined criterion. Such cloud computing may be provided by cloud computing platforms such as, for example, Amazon Web Services (AWS), Microsoft Azure, Google Cloud Platform, and IBM cloud. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, generated sample fingerprints comprising quantitative measures of nucleic acid molecules at each of a plurality of genetic loci, determined differences between two sample fingerprints, and identified sample mismatches. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, process nucleic acid molecules to generate a sample fingerprint comprising a quantitative measure of the nucleic acid molecules at each of a plurality of genetic loci, determine a difference between two sample fingerprints, and identify a sample mismatch when the difference between two sample fingerprints satisfies a predetermined criterion.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. (canceled)

2. A method for identifying a sample mismatch, comprising:
obtaining a first biological sample comprising a first plurality of nucleic acid molecules from a subject;
processing, by a computer, the first plurality of nucleic acid molecules to generate a first sample fingerprint comprising a quantitative measure of the first plurality of nucleic acid molecules at each of a plurality of genetic loci, wherein the plurality of genetic loci comprises autosomal single nucleotide polymorphisms (SNPs);
obtaining a second biological sample comprising a second plurality of nucleic acid molecules from the subject;
processing, by a computer, the second plurality of nucleic acid molecules to generate a second sample fingerprint comprising a quantitative measure of the second plurality of nucleic acid molecules at each of the plurality of genetic loci;
determining a difference between the first sample fingerprint and the second sample fingerprint; and
identifying the sample mismatch when the difference between the first sample fingerprint and the second sample fingerprint exceeds a predetermined threshold,
wherein the autosomal single nucleotide polymorphisms comprise simple single nucleotide polymorphisms.

3. (canceled)

4. The method of claim 2, wherein the autosomal single nucleotide polymorphisms have a minor allele fraction that exceeds about 7.5%.

5. The method of claim 2, wherein the first plurality of nucleic acid molecules and the second plurality of nucleic acid molecules comprise cell-free DNA (cfDNA), buffy coat DNA, or solid tumor DNA.

6. (canceled)

7. (canceled)

8. The method of claim 2, wherein the second biological sample is obtained from the subject at a later time after obtaining the first biological sample.

9. The method of claim 2, wherein processing the first plurality of nucleic acid molecules comprises sequencing the first plurality of nucleic acid molecules to generate a first plurality of sequencing reads, and wherein processing the second plurality of nucleic acid molecules comprises sequencing the second plurality of nucleic acid molecules to generate a second plurality of sequencing reads.

10. The method of claim 9, wherein the sequencing comprises whole genome sequencing (WGS).

11. The method of claim 10, wherein the sequencing is performed at a depth of no more than about 10×.

12. (canceled)

13. (canceled)

14. The method of claim 9, wherein the quantitative measure of the first plurality of nucleic acid molecules comprises a coverage of the first plurality of nucleic acid molecules at each of the plurality of genetic loci, and wherein the quantitative measure of the second plurality of nucleic acid molecules comprises a coverage of the second plurality of nucleic acid molecules at each of the plurality of genetic loci.

15. The method of claim 2, wherein processing the first plurality of nucleic acid molecules comprises performing binding measurements of the first plurality of nucleic acid molecules, and wherein processing the second plurality of nucleic acid molecules comprises performing binding measurements of the second plurality of nucleic acid molecules.

16. The method of claim 15, wherein the quantitative measure of the first plurality of nucleic acid molecules at each of the plurality of genetic loci comprises a number of the first plurality of nucleic acid molecules containing the genetic locus, and wherein the quantitative measure of the second plurality of nucleic acid molecules at each of the plurality of genetic loci comprises a number of the second plurality of nucleic acid molecules containing the genetic locus.

17. The method of claim 2, further comprising enriching the first plurality of nucleic acid molecules and/or the second plurality of nucleic acid molecules for at least a portion of the plurality of genetic loci.

18. The method of claim 17, wherein the enrichment comprises amplifying at least a portion of the first plurality of nucleic acid molecules and/or the second plurality of nucleic acid molecules.

19. The method of claim 18, wherein the amplification comprises selective amplification or universal amplification.

20. (canceled)

21. The method of claim 17, wherein the enrichment comprises selectively isolating at least a portion of the first plurality of nucleic acid molecules and/or the second plurality of nucleic acid molecules.

22. The method of claim 2, wherein the plurality of genetic loci comprises at least about 50 distinct autosomal single nucleotide polymorphisms (SNPs).

23. (canceled)

24. The method of claim 2, wherein generating the first sample fingerprint further comprises obtaining a third biological sample comprising a third plurality of nucleic acid molecules from the subject, and processing the third plurality of nucleic acid molecules to obtain a quantitative measure of the third plurality of nucleic acid molecules at each of a second plurality of genetic loci, wherein the second plurality of genetic loci comprises autosomal single nucleotide polymorphisms (SNPs); and wherein generating the second sample fingerprint further comprises obtaining a fourth biological sample comprising a fourth plurality of nucleic acid molecules from the subject, and processing the fourth plurality of nucleic acid molecules to obtain a quantitative measure of the fourth plurality of nucleic acid molecules at each of the second plurality of genetic loci.

25-27. (canceled)

28. The method of claim 24, wherein generating the first sample fingerprint further comprises obtaining a fifth biological sample comprising a fifth plurality of nucleic acid molecules from the subject, and processing the fifth plurality of nucleic acid molecules to obtain a quantitative measure of the fifth plurality of nucleic acid molecules at each of a third plurality of genetic loci, wherein the third plurality of genetic loci comprises autosomal single nucleotide polymorphisms (SNPs); and wherein generating the second sample fingerprint further comprises obtaining a sixth biological sample comprising a sixth plurality of nucleic acid molecules from the subject, and processing the sixth plurality of nucleic acid molecules to obtain a quantitative measure of the sixth plurality of nucleic acid molecules at each of the third plurality of genetic loci.

29-31. (canceled)

32. The method of claim 2, comprising identifying the sample mismatch with a sensitivity or specificity of at least about 90%.

33. (canceled)

34. The method of claim 2, comprising identifying the sample mismatch with a positive predictive value (PPV) of at least about 90%, a negative predictive value (NPV) of at least about 90%, or an area under the curve (AUC) of at least about 0.90.

35. (canceled)

36. (canceled)

37. The method of claim 2, wherein the predetermined criterion threshold is that the difference comprises a difference in genotype similarity greater than a predetermined threshold.

38. The method of claim 37, wherein the predetermined threshold is about 0.8.

39. The method of claim 2, further comprising excluding the second biological sample from further assaying based on the identified sample mismatch.

40. The method of claim 2, further comprising identifying a sample match when the difference between the first sample fingerprint and the second sample fingerprint does not satisfy the predetermined threshold.

41. The method of claim 40, comprising identifying the sample match with a sensitivity of at least about 90%, a specificity of at least about 90%, a positive predictive value (PPV) of at least about 90%, a negative predictive value (NPV) of at least about 90%, or an area under the curve (AUC) of at least about 0.90.

42-45. (canceled)

46. The method of claim 40, further comprising: (a) subjecting the second biological sample to further assaying based on the identified sample match; or (b) based on the identified sample match, storing the second sample fingerprint in a database, and optionally, storing the first sample fingerprint in the database.

47. (canceled)

48. A non-transitory computer-readable medium comprising machine-executable code that, upon execution by one or more computer processors, implements a method for identifying a sample mismatch, comprising:
receiving information of a first sample fingerprint comprising a quantitative measure of a first plurality of nucleic acid molecules of a first biological sample at each of a plurality of genetic loci, wherein the plurality of genetic loci comprises autosomal single nucleotide polymorphisms (SNPs) that comprise simple single nucleotide polymorphisms;
receiving information of a second sample fingerprint comprising a quantitative measure of a second plurality of nucleic acid molecules of a second biological sample at each of the plurality of genetic loci, wherein the second biological sample is obtained from the subject;
determining a difference between the first sample fingerprint and the second sample fingerprint; and
identifying the sample mismatch when the difference between the first sample fingerprint and the second sample fingerprint satisfies a predetermined threshold.

49. The method of claim 2, wherein the quantitative measure of the first plurality of nucleic acid molecules comprises no more than twelve independent measurements of the first plurality of nucleic acid molecules.

50. The method of claim 2, wherein the autosomal single nucleotide polymorphisms have a minor allele fraction that exceeds a predetermined threshold.

51. A system, comprising:
one or more processors;
a non-transitory computer-readable medium comprising machine-executable code that, upon execution by the one or more processors, implements a method for identifying a sample mismatch, comprising:
receiving information of a first sample fingerprint comprising a quantitative measure of a first plurality of nucleic acid molecules of a first biological sample at each of a plurality of genetic loci, wherein the plurality of genetic loci comprises autosomal single nucleotide polymorphisms (SNPs) that comprise simple single nucleotide polymorphisms;
receiving information of a second sample fingerprint comprising a quantitative measure of a second plurality of nucleic acid molecules of a second biological sample at each of the plurality of genetic loci, wherein the second biological sample is obtained from the subject;
determining a difference between the first sample fingerprint and the second sample fingerprint; and
identifying the sample mismatch when the difference between the first sample fingerprint and the second sample fingerprint satisfies a predetermined threshold.
